(19) United States
(12) Patent Application Publication
(10) Pub. No.: US 2014/0276137 A1
(43) Pub. Date: Sep. 18, 2014

(54) SYSTEMS AND METHODS FOR DETERMINING CORONARY FLOW RESERVE

(71) Applicant: VOLCANO CORPORATION, San Diego, CA (US)

(72) Inventors: Joseph Burnett, Carlsbad, CA (US); Bret Millett, Folsom, CA (US)

(73) Assignee: VOLCANO CORPORATION, San Diego, CA (US)

(21) Appl. No.: 14/205,941

(22) Filed: Mar. 12, 2014

Related U.S. Application Data
(60) Provisional application No. 61/782,205, filed on Mar. 14, 2013.

(51) Int. Cl. A61B 5/02; A61B 5/0215

(52) U.S. Cl. CPC A61B 5/02; A61B 5/0215
USPC 600/486; 600/481; 600/485

(57) ABSTRACT

The invention generally relates to systems and methods for determining coronary flow reserve (CFR). The invention provides systems and methods for determining coronary flow reserve using a flow reserve index obtained at rest and during hyperemia. One flow reserve index obtained under the two conditions can be used to compute coronary flow reserve. The difference between the resting value and the hyperemic value of the index correlates to a coronary flow reserve value.

### CROSS-REFERENCE TO RELATED APPLICATION

This application claims the benefit of, and priority to, U.S. Provisional Patent Application No. 61/782,205, filed Mar. 14, 2013, which is incorporated by reference.

### FIELD OF THE INVENTION

The invention generally relates to determining coronary flow reserve.

### BACKGROUND

The human heart works through the action of heart muscle known as myocardium. The myocardium receives and uses oxygen from blood delivered by coronary arteries. The deoxygenated blood is removed by cardiac veins. The coronary arteries provide the flow of blood that is required for the heart to beat. Coronary arteries can get narrowed and be blocked by atherosclerotic plaque. A narrowing of the arteries is called a stenosis. Coronary stenosis impedes the flow of blood to the heart, and can result in angina or a heart attack.

Coronary flow reserve (CFR) refers to the body's ability to increase blood flow to the heart when needed. Since coronary stenosis impedes the flow of blood to the heart, CFR is a potential indicator of severity of coronary stenosis. The ability to identify a poor CFR could aid doctors in preventing heart attacks.

### SUMMARY

The invention provides systems and methods for determining coronary flow reserve using a flow reserve index obtained at rest and during hyperemia. One flow reserve index obtained under the two conditions can be used to compute coronary flow reserve. The difference between the resting value and the hyperemic value of the index correlates to a coronary flow reserve value. If the resting value and the hyperemic value are obtained by a computer system such as an imaging engine of a medical imaging system, the system can automatically apply an algorithm to the resting value and the hyperemic value and obtain a CFR value. The system can then automatically provide the CFR value, optionally with a confidence measure. The provided CFR value could be used with the measured indexes to classify severity of a lesion or stenosis by including both predicted flow and pressure information, instead of using only ones of those data points taken separately and alone. This allows a doctor to evaluate the severity of a condition more robustly, giving more precise information about the patient's degree of coronary stenosis and risk of heart attack. Treatment decisions can be based on the evaluation, to provide the patient with relief from the condition.

In certain aspects, the invention provides a method of determining coronary flow reserve, the method that includes obtaining a resting value for a flow reserve index from a patient, obtaining a hyperemic value for the flow reserve index from the patient, computing the coronary flow reserve based on the resting value and the hyperemic value, and providing the coronary flow reserve to a user. The index may be fractional flow reserve (e.g., based on a mean aortic pressure and a mean distal coronary pressure, e.g., Pd/Pa), an instantaneous wave-free ratio (iFR), or any other suitable index.

The resting value and the hyperemic value may be obtained using an intravascular pressure sensor. Preferably obtaining the resting value and the hyperemic value comprises receiving the resting value and the hyperemic value at a computer system comprising a tangible, non-transitory memory coupled to a processor. The coronary flow reserve, optionally along with the resting value and the hyperemic value, may be displayed on a monitor.

In some embodiments, the coronary flow reserve is provided while the intravascular pressure sensor is inserted into a vessel of the patient or the resting value and the hyperemic value are obtained by inserting any suitable medical instrument (e.g., an intravascular probe) into the patient.

In certain embodiments, the method further includes suggesting a diagnosis of a cardiovascular condition based on the coronary flow reserve. A positive diagnosis may be suggested if the coronary flow reserve is less than a critical value (e.g., 2).

In related aspects, the invention provides a medical intervention system operable to perform any combination of the steps described above.

### BRIEF DESCRIPTION OF THE DRAWINGS

FIG. 1 depicts a system for determining coronary flow reserve.

FIG. 2 shows determining coronary flow reserve.

FIG. 3 diagrams measurement points.

FIG. 4 illustrates a measurement result.

FIG. 5 charts a method of determining coronary flow reserve.

FIG. 6 shows a system of the invention.

### DETAILED DESCRIPTION

The invention provides systems and methods by which coronary flow reserve (CFR) may be determined. Coronary flow reserve describes the ability of coronary blood flow to increase substantially when metabolic requirements demand it. See Nitenberg and Antony, 1995, Coronary vascular reserve in humans: a critical review, Eur Heart J 16 Suppl I-7-21; Collins, 1993, Coronary flow reserve, B Heart J 69:279-281; and Gould, et al., 1974, Physiologic basis for assessing critical coronary stenosis, Am J Cardiol 33:87-94, the contents of each of which are incorporated by reference. In some embodiments, CFR is determined using a medical system.

FIG. 1 depicts a system101 for determining coronary flow reserve. System 101 includes a host workstation 110 coupled to a catheter 112 having a probe 114 on the end. The catheter 112 may optionally be controlled by an interface module 105. Catheter insertion may be guided by angiographic procedures using c-arm 107. Measurement results can displayed on a monitor 103. Data may be processed within host workstation 110, or using a connected remote terminal or server 120.

FIG. 2 shows determining coronary flow reserve. Here, a physician uses probe 112 to measure a flow index. Preferably, Pd/Pa is measured. Optional interface module 105 may be used to control probe 112. An imaging engine 150 in host workstation 110 obtains measured index values and calculates CFR as described herein. The CFR and measured index values may be displayed on monitor 103.

Fractional Flow Reserve (FFR) represents maximum achievable flow, which determines the functional capacity of a patient. At maximum vasodilation (e.g., corresponding to maximum hyperemia), blood flow to the myocardium is proportional to myocardial perfusion pressure.

FFR may be measured by placing a pressure measuring guide wire directly in the coronary artery while adenosine (a pharmacologic agent used to increase coronary blood flow) is administered to the patient. In some embodiments, regadenoson is used in place of adenosine. The ratio of the blood pressure in the diseased vessel to the blood pressure in the aorta at the origin of the coronary artery is the FFR value. FFR has been recognized as a “gold standard” for guiding treatment for coronary artery disease.

Fractional flow reserve (FFR) is a criteria typically used to assess blood flow. Fractional flow reserve is determined by measuring maximum flow in the presence of a stenosis (i.e., a narrowing of the blood vessel) divided by normal maximum flow. This ratio is approximately equal to the mean hyperemic (i.e., dilated vessel) distal coronary pressure divided by the mean aortic pressure. Distal coronary pressure is usually measured with a pressure sensor mounted on the distal portion of a guidewire after administering a hyperemic agent into the blood vessel. Mean aortic pressure is measured using a variety of techniques in areas proximal of the stenosis, for example, in the aorta.

FFR provides a convenient, cost-effective way to assess the severity of coronary and peripheral lesions. FFR also provides an index of stenosis severity that allows rapid determination of whether a blockage is significant enough to limit blood flow within the artery, thereby requiring treatment. The normal value of FFR is about 1.00. Values less than 0.80 are deemed significant and require treatment, which may include angioplasty and stenting.

As encompassed by the invention, a baseline FFR measurement is taken prior to conducting the therapeutic procedure. The procedure is then performed, and a subsequent post-therapy FFR measurement is taken. The post-therapy measurement is compared to the baseline measurement, and the degree in improvement is ascertained. As described herein, the degree of improvement resulting from the therapy is known as functional gain. For example, the FFR of an apparently occluded blood vessel is ascertained to be 0.75. As this is below the threshold value for therapeutic intervention, the patient will receive a stent to restore flow in the vessel. After the stent procedure, FFR is again assessed in the area of interest. This time, the FFR is determined to be 0.97. Comparing the second FFR reading to the first, the patient has a functional gain of 29%. While the second FFR determination does indicate that the operation is a success, (the blood flow is now essentially at normal levels), it does not quantify the degree of success. Methods of the invention provide just that, the ability to determine and document the degree of improvement after a therapeutic procedure has been performed. Accordingly, methods of the invention provide highly practical tools to monitor a patient's progress after therapeutic intervention.

Determination of FFR typically involves the insertion of a pressure sensing guidewire into a blood vessel and measuring pressure inside the vessel with the device. The actual parameters and calculations for determining FFR are well known in the art and are described above.

In practice, measuring pressure inside the vessel may also involve injecting a local anesthetic into the skin to numb the area of the patient prior to surgery. A puncture is then made with a needle in either the femoral artery of the groin or the radial artery in the wrist before the provided guidewire is inserted into the arterial puncture. Once positioned, the guidewire may then be used to measure pressure in the vessel, and subsequently FFR.

In a typical procedure, the guidewire may be advanced to a location on the distal side of the stenosis. The pressure may then be measured at a first flow state. Then, the flow rate may be significantly increased, for example by the use of drugs such as adenosine, and the pressure measured in this second, hyperemic, flow state. The pressure and flow relationships at these two flow states are then compared to assess the severity of the stenosis and provide improved guidance for any coronary interventions. As explained above, FFR is a comparison of the pressure within a vessel at positions prior to the stenosis and after the stenosis. The level of FFR determines the significance of the stenosis, which allows physicians to more accurately identify clinically relevant stenosis. For example, an FFR measurement above 0.80 indicates normal coronary blood flow and a non-significant stenosis. A measurement below 0.80 indicates the necessity of therapeutic intervention

Any medical device can be used in conjunction with the provided methods for taking physiological measurements (e.g., FFR), before and after a therapeutic procedure. In certain embodiments, the device is configured for insertion into a bodily lumen, such as a guidewire or catheter. In other embodiments, the medical device is a pressure-sensing guidewire or catheter. In additional embodiments, the medical device is flow-sensing guidewire or catheter. In further embodiments, the encompassed guidewire or catheter has both flow and pressure measuring capabilities.

FIG. 3 illustrates an artery 301. Pa is the mean aortic pressure at maximum hyperemia. Pd is the mean distal coronary pressure at maximum hyperemia. Pv is venous pressure. Pressure may be measured using a pressure wire by inserting and calibrating the wire, equalizing, advancing wire through stenosis and inducing hyperemia. Systems and methods of the invention may be used to predict CFR by measuring the difference between resting Pd/Pa and hyperemic Pd/Pa. A delta between the two is positively correlated to a CFR value. Systems and methods of the invention may be used to automatically record the resting Pd/Pa or iFR and the hyperemic Pd/Pa or iFR; calculate the difference; and measure against a clinically built look-up table to show what the predicted CFR value would be, as well as the confidence interval on that number. That number, along with the pressure measurement at rest and hyperemia, may be used to classify lesion severity by having both predicted flow and pressure data.

FIG. 4 shows a monitor 103 showing results of the pressure measurement. FFR normalizes to 1 for all patients. FFR accounts for the interaction between epicardial stenosis severity and extent of perfusion territory. Measuring CFR is discussed in U.S. Pat. No. 6,094,591 to Foltz and U.S. Pub. 2003/0032886 to Dgany, the contents of each of which are incorporated by reference.

It is understood that FFR is predicted by Pd/Pa, e.g., by FFR=Pa/Pd. See Mams, et al, 2010, Resting Pd/Pa measured with intracoronary pressure wire strongly predicts fractional flow reserve, J Invasive Cardiol 22(6):260-265; Tonino et al., 2009, Fractional flow reserve versus angiography for guiding percutaneous coronary intervention, N Engl J Med 360:213-224.

In some embodiments, FFR is computed via a non-invasive method. For example, a cardiac CT scan can be used to determine blood pressure and blood flow throughout the coronary tree. This allows computation of FFR by CT (FFRCT). The FFRCT computation may be provided by HeartFlow, Inc. (Redwood City, Calif.). See Koo et al, 2011, Diagnosis of Ischemia-Causing Coronary Stenoses by Noninvasive Fractional Flow Reserve Computed From Coronary Computed Tomographic Angiograms, J Am Coll Cardiol 58(19):1989-1997.

In some embodiments, an index of stenosis is used in a fashion similar to the FFR. See Sen, et al., 2012, Development and validation of a new adenosine-independent index of stenosis severity from coronary wave-intensity analysis, J Am Coll Cardiol 59(15):1392-1402.

The instantaneous wave-free ratio (iFR) is a an index of stenosis derived from pressure measurements described by Sen, et al. (2012). The iFR index uses pressure measurements obtained selectively at a specific time interval of the cardiac cycle. That interval is a diastolic interval in which intracoronary resistance at rest is equivalent to time-averaged resistance during FFR measurements.

Sen et al. (2012) proposed that pressure measurements obtained selectively at that specific interval can be used to obtain the iFR. Changes in coronary hemodynamics over the cardiac cycle are assessed by calculating instantaneous resistance and by applying wave-intensity analysis. An index of resistance is calculated as the ratio between pressure and flow velocity. Wave-intensity is analyzed to identify wave-free periods. Briefly, onset of diastole may be identified from the dicrotic notch in a backward-traveling wave, and the diastolic window is calculated beginning 25% of the way into diastole and ending 5 ms before the end of diastole. Mean pressure distal to the stenosis during the diastolic wave-free period (Pd wave-free period) divided by the mean aortic pressure during the diastolic wave-free period (Pa wave-free period) gives iFR. See Sen, et al. (2012). During this wave-free period, the onset of minimal resistance is identified and its value calculated. Mean intracoronary resistance and its coefficient of variation may be calculated over a minimum of 3 beats. Intracoronary and intravenous adenosine and pressure wires can be obtained from Volcano. Calculation of iFR may be done with a fully automated Matlab algorithm (Mathworks, Inc.) as described in Sen, et al. (2012).

FIG. 5 charts a method of determining coronary flow reserve. Preferably, a resting index is obtained such as Pd/Pa at rest. Hyperemia is induced (e.g., by an infusion of adenosine). A hyperemic index is then obtained. These indices are relayed into work station 110 or another computer component of system 101. An algorithm is applied to calculate CFR. In some embodiments, the algorithm includes calculating CFR=k((Pd/Pa)hyperemic−(Pd/Pa)rest), where k is a constant determined by sampling a population with empirically known CFR values. In some embodiments, the algorithm includes calculating CFR=k((iFR)hyperemic−(iFR)rest), where k is a constant determined by sampling a population with empirically known CFR values. The CFR is then provided (e.g., by display on monitor 103).

FIG. 6 shows a system 600 of the invention. Preferably the system includes as work station 110 a computer device. A computer device will generally include a tangible, non-transitory memory 617 coupled to a processor 609 and an input/output mechanism 605. Display monitor 103 is one example of I/O 605. Imaging engine 150 may be coupled to workstation 110 and to the detection probe 112. System 600 may optionally include a terminal or server computer 409, and components of system 600 may be coupled by communication network 409.

System 600 may include components obtained from Volcano Corporation (San Diego, Calif.). System 600 may include components described in U.S. Pat. No. 8,277,386 to Ahmed; U.S. Pat. No. 5,873,835 to Hastings; U.S. Pat. No. 5,313,957 to Little; U.S. Pat. No. 4,928,693 to Goodin; U.S. Pub. 2012/0271170 to Emelianov; and U.S. Pub. 2012/0220865 to Brown, the contents of each of which are incorporated by reference.

As used herein, the word “or” means “and or or”, sometimes seen or referred to as “and/or”, unless indicated otherwise.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of determining coronary flow reserve, the method comprising:
obtaining a resting value for a flow reserve index from a patient;
obtaining a hyperemic value for the flow reserve index from the patient;
computing the coronary flow reserve based on the resting value and the hyperemic value; and
providing the coronary flow reserve to a user.

2. The method of claim 1, wherein the flow reserve index comprises a fractional flow reserve.

3. The method of claim 1, wherein the flow reserve index comprises an instantaneous wave-free ratio.

4. The method of claim 1, wherein the flow reserve index comprises a mean aortic pressure at maximum hyperemia (Pa) and a mean distal coronary pressure at maximum hyperemia (Pd).

5. The method of claim 4, wherein the flow reserve index comprises Pd/Pa.

6. The method of claim 5, wherein the resting value and the hyperemic value are obtained using an intravascular pressure sensor.

7. The method of claim 6, wherein obtaining the resting value and the hyperemic value comprises receiving the resting value and the hyperemic value at a computer system comprising a tangible, non-transitory memory coupled to a processor.

8. The method of claim 7, wherein providing the coronary flow reserve comprises displaying the coronary flow reserve on a monitor.

9. The method of claim 8, wherein providing the coronary flow reserve comprises displaying the coronary flow reserve, the resting value, and the hyperemic value on the monitor.

10. The method of claim 9, wherein the coronary flow reserve is provided while the intravascular pressure sensor is inserted into a vessel of the patient.

11. The method of claim 1, wherein the coronary flow reserve is provided while an intravascular pressure sensor is inserted into a vessel of the patient.

12. The method of claim 1, wherein the resting value and the hyperemic value are obtained by inserting a medical instrument into the patient.

13. The method of claim 12, wherein obtaining the resting value and the hyperemic value further comprises using the medical instrument to measure pressure.

14. The method of claim 12, wherein the medical instrument comprises an intravascular probe.

15. The method of claim 1, further comprising suggesting a diagnosis of a cardiovascular condition based on the coronary flow reserve.

16. The method of claim 15, wherein a positive diagnosis is suggested if the coronary flow reserve is less than a critical value.

17. The method of claim 16, wherein the critical value is 2.

18. The method of claim 9, further comprising suggesting a diagnosis of a cardiovascular condition based on the coronary flow reserve.

19. The method of claim 18, wherein a positive diagnosis is suggested if the coronary flow reserve is less than a critical value.

20. The method of claim 19, wherein the critical value is 2.
